(19) United States
(12) Patent Application Publication
(10) Pub. No.: US 2008/0139525 A1
(43) Pub. Date: Jun. 12, 2008

(54) ANTIOXIDANT THERAPIES

(75) Inventor: Joseph Loscalzo, Dover, MA (US)

Correspondence Address: WOLF GREENFIELD & SACKS, P.C., 600 ATLANTIC AVENUE, BOSTON, MA, 02210-2206, US

(73) Assignee: The Brigham and Women's Hospital, Inc., Boston, MA (US)

(21) Appl. No.: 11/848,393

(22) Filed: Aug. 31, 2007

Related U.S. Application Data
(60) Provisional application No. 60/841,654, filed on Aug. 31, 2006.

(51) Int. Cl. A61K 31/555; A61P 29/00

(52) U.S. Cl. 514/185

(57) ABSTRACT

The invention relates, in part, to methods of treatment of inflammatory, autoimmune, vascular and cardiovascular conditions with a combination of a superoxide dismutase (SOD) mimetic and a selenium (Se) compound. The invention also relates to compositions comprising a SOD mimetic(s) and a Se compound.

### RELATED APPLICATIONS

This application claims priority under 35 U.S.C. §119(e) from U.S. Provisional Application Ser. No. 60/841654, filed Aug. 31, 2006, and entitled: ANTIOXIDANT THERAPIES incorporated herein in its entirety by reference.

### FIELD OF THE INVENTION

The invention relates to antioxidant therapies for inflammatory, autoimmune, vascular and cardiovascular conditions.

### BACKGROUND OF THE INVENTION

Oxidative stress is imposed on cells as a result of one or a combination of any of three factors: 1) an increase in reactive oxygen species (ROS) generation, 2) a decrease in antioxidant protection, or 3) a failure to repair oxidative damage. Cell damage is induced by ROS. ROS are free radicals, reactive anions containing oxygen atoms, or molecules containing oxygen atoms that can either produce free radicals or are chemically activated by them. Examples of ROS include hydroxyl radical, superoxide, hydrogen peroxide, and peroxynitrite. The main source of ROS in vivo is aerobic respiration, although ROS are also produced by peroxisomal β-oxidation of fatty acids, microsomal cytochrome P450 metabolism of xenobiotic compounds, stimulation of phagocytosis by pathogens or lipopolysaccharides, arginine metabolism, and tissue specific enzymes. Under normal conditions, ROS are cleared from the cell by the action of superoxide dismutase (SOD), catalase, or glutathione (GSH) peroxidase.

Oxidative stress and ROS have been implicated in many conditions and the number of conditions associated with oxidative stress continues to increase. Oxidative stress serves either as the mechanistic basis of these conditions or as a secondary basis for their complications. See, for example, Simic, M. G. et al., Basic Life Sciences, Vol. 49, Plenum Press, New York and London, 1988; Petkau, A., Cancer Treat. Rev. 13, 17 (1986); McCord, J. Free Radicals Biol. Med., 2, 307 (1986); and Bannister, J. V. et al., Crit. Rev. Biochem., 22, 111 (1987).

The involvement of ROS in disease derives mostly from work on superoxide. The knowledge about the role of superoxide in disease has been gathered using the native SOD enzyme and by the use of SOD knockout models or transgenic models that overexpress the various isoforms of the enzyme. Although the native enzyme has shown promising anti-inflammatory properties in both preclinical and clinical studies, there were drawbacks associated with its use as a therapeutic agent. Based on the concept that removal of superoxide modulates the course of inflammation, synthetic, low-molecular-weight mimetics of the SOD enzymes that could overcome some of the limitations associated with the use of the native enzyme have been designed. Advances made using various SOD mimetics led to the proposal that superoxide (and/or the product of its interaction with nitric oxide, peroxynitrite) is an important mediator of inflammation, and to the conclusion that SOD mimetics can be utilized as therapeutic agents in conditions of various etiologies.

Selenium (Se) compounds have also been utilized as antioxidants (or as oxygen radical scavengers) in the treatment of numerous conditions associated with oxidative stress to protect cells against the effects of oxygen radicals. Se, Se metabolites and selenoproteins have been identified to be involved in numerous cellular processes. One of the most important biological functions of Se is attributed to its presence in the enzyme GSH peroxidase. The availability of Se and the synthesis of selenocysteine for incorporation into the GSH peroxidases is important for the local antioxidant potential in cells and is crucial in the cellular defense mechanism against superoxide radicals.

Major antioxidant therapy trials to date using SOD mimetics or Se compounds, have failed to show a benefit in hard clinical endpoints. This lack of benefit may either be a reflection of the duration of therapy, the dose of antioxidant (especially as higher doses of commonly used nonspecific antioxidants can redox couple with select biomolecules to promote oxidation), or the lack of targeting (molecularly or compartmentally) of the agent used, rather than a flaw in the plausibility of the oxidative stress hypothesis.

Thus, there is a strong incentive to identify new and/or better antioxidant treatment options for conditions associated with oxidative stress such as inflammatory, autoimmune, vascular and cardiovascular conditions.

### SUMMARY OF THE INVENTION

The invention is based, in part, on the identification of antioxidant combinations that address oxidant stress targets in the cell. Applicant has surprisingly and unexpectedly found that SOD mimetics used in combination with Se compounds resulted in an enhanced and synergistic inhibition of ROS generation.

Without intending to be bound by any particular mechanism or theory, it is believed that SOD mimetics, by minimizing the flux of superoxide, and Se compounds, by increasing the expression of the GSH peroxidases, work synergistically to minimize overall ROS generation.

The present invention provides methods and compositions for treating inflammatory, autoimmune, vascular and cardiovascular conditions.

According to one aspect of the invention, a method for treating a subject having an inflammatory condition is provided. The method comprises administering an effective amount of a SOD mimetic and a Se compound to the subject to treat the subject. SOD mimetics include, for example, manganese-tetrakis (4-benzoic acid) porphyrin (MnTBAP), M40403, manganese metalloporphyrins, manganese salen complexes, nitroxides, and manganese (II) (pentaazamacrocyclic ligand)-based complexes. Examples of Se compounds include sodium selenite, selenomethionine, methylselenocysteine, 2-methyl-selenoazolidine, selenobetaine methyl ester, selenocysteine, selenobetaine, and selenoazolidine.

Either or both of the SOD mimetic and the Se compound may be administered orally, intravenously, intramuscularly, sublingually, buccally, intranasally, intraarticularly, intraperitoneally, subcutaneously, or topically. In some embodiments the SOD mimetic and the Se compound are administered prophylactically.

Examples of inflammatory conditions include allergic rhinitis, ankylosing spondilitis, arthritis, asthma, Behcet syndrome, bursitis, chronic obstructive pulmonary disease (COPD), Churg-Strauss syndrome, dermatitis, gout, Henoch-Schonlein purpura, inflammatory bowel disease (Crohn's disease or ulcerative colitis), inflammatory neuropathy, Kawasaki disease, myositis, neuritis, pericarditis, polyarteritis nodosa, polymyalgia rheumatica, prostatitis, psoriasis, radiation injury, sarcoidosis, shock, systemic inflammatory response syndrome (SIRS), Takayasu's arteritis, temporal arteritis, thromboanginitis obliterans (Buerger's disease), vasculitis, and Wegener's granulomatosus.

In some embodiments, the method further comprises administering an anti- inflammatory agent that is not a SOD mimetic or a Se compound. Examples anti-inflammatory agents that are not SOD mimetics or Se compounds are listed below.

According to another aspect of the invention, a method for treating a subject having an autoimmune condition is provided. The method comprises administering an effective amount of a SOD mimetic and a Se compound to the subject to treat the subject.

Examples of autoimmune conditions include Addison's disease, chronic thyroiditis, dermatomyositis, Grave's disease, Hashimoto's thyroiditis, hypersensitivity pneumonitis, insulin-dependent diabetes mellitus (type I diabetes), insulin-independent diabetes mellitus (type II diabetes), multiple sclerosis, myasthenia gravis, organ transplantation, pernicious anemia, Reiter's syndrome, rheumatoid arthritis, Sjogren's syndrome, systemic lupus erythematosis (SLE), thyroiditis, and urticaria.

In some embodiments, the method further comprises administering an autoimmune condition agent that is not a SOD mimetic or a Se compound. In some embodiments, the autoimmune condition agent may be, for example, an immunosuppressive agent. Examples of immunosuppressive agents are listed below.

According to another aspect of the invention a method for treating a subject having a vascular or a cardiovascular condition or is at risk of developing a cardiovascular condition is provided. The method comprises administering an effective amount of a SOD mimetic and a Se compound to the subject to treat the subject. In some important embodiments, the subject has the vascular or the cardiovascular condition.

Examples of vascular conditions include diabetic retinopathy, diabetic nephropathy, renal fibrosis, hypertension, atherosclerosis, arteriosclerosis, atherosclerotic plaque, atherosclerotic plaque rupture, cerebrovascular accident (stroke), transient ischemic attack (TIA), peripheral artery disease, arterial occlusive disease, vascular aneurysm, ischemia, ischemic ulcer, heart valve stenosis, heart valve regurgitation and intermittent claudication.

Examples of cardiovascular conditions include coronary artery disease, ischemic cardiomyopathy, myocardial ischemia, and ischemic or post-myocardial ischemia revascularization.

In some embodiments the method further comprises administering a vascular agent or a cardiovascular agent that is not a SOD mimetic or a Se compound. Examples of vascular agents and cardiovascular agents that are not SOD mimetics or Se compounds are listed below.

According to another aspect of the invention a pharmaceutical composition is provided. The pharmaceutical composition comprises a SOD mimetic, a Se compound, and a pharmaceutically acceptable carrier. In some embodiments, the SOD mimetic and the Se-containing compound are present in an effective amount. The pharmaceutical composition may further comprise an agent that is not a SOD mimetic or a Se compound. The agent may be an antioxidant agent that is not a SOD mimetic or a Se compound. Examples of antioxidant agents that are not SOD mimetics or Se compounds include but are not limited to Vitamin A, Vitamin C, Vitamin E, ubiquinone (Coenzyme Q-10), allopurinol, alpha lipoic acid (ALA), oxothiazolidine-4-carboxylate, glutathione monoethylester and carotenoids such as alphacarotene, betacarotene, lycopene, lutein, cryptoxanthin and zeaxanthin. The agent that is not a SOD mimetic or a Se compound may be an anti-inflammatory agent, an immunosuppressive agent, a vascular or a cardiovascular agent.

According to another aspect of the invention a composition is provided. The composition comprises manganese-tetrakis (4-benzoic acid) porphyrin (MnTBAP) in a unit dosage of about 350 mg and a Se-methylselenocysteine in a unit dosage of about 150 mg. In some important embodiments, the composition comprises a pharmaceutically acceptable carrier.

According to another aspect of the invention a kit is provided. The kit comprises a SOD mimetic and a Se compound and instructions of use.

These and other aspects of the invention, as well as various advantages and utilities, will be more apparent with reference to the detailed description of the invention. Each aspect of the invention can encompass various embodiments as will be understood.

### BRIEF DESCRIPTION OF THE DRAWINGS

FIG. 1 is an outline of the central oxidant pathway.

FIG. 2 is an outline of the biochemical fates of ROS.

FIG. 3 shows the structures of the indicated of Se compounds.

FIG. 4 is a schematic representation of a kit according to the invention.

FIG. 5 are two histograms showing the effect of no-flow ischemia followed by reperfusion with or without a solution containing 23 μM of the SOD mimetic M40403 alone or in combination with dietary Se on EDP and DevP in wild-type (WT) rats hearts compared to rats hearts where glutathione peroxidase-1, the selenocysteine-containing antioxidant protein, was overexpressed as a transgene (GPx-TG) (n=3−5; *: p<0.02).

FIG. 6 are two histograms showing the effect of no-flow ischemia followed by reperfusion with or without a solution containing 23 μM of the SOD mimetic M40403 alone or in combination with Se supplementation (as sodium selenite, 50 μg/kg/day) administered for 2 weeks on EDP and DevP in rats hearts (*: p<0.04).

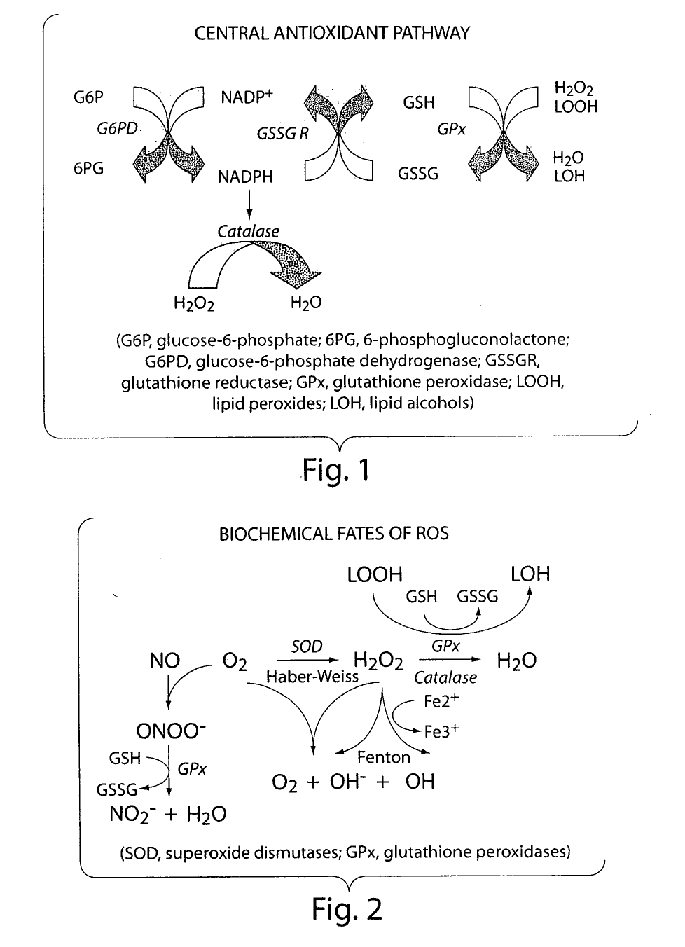

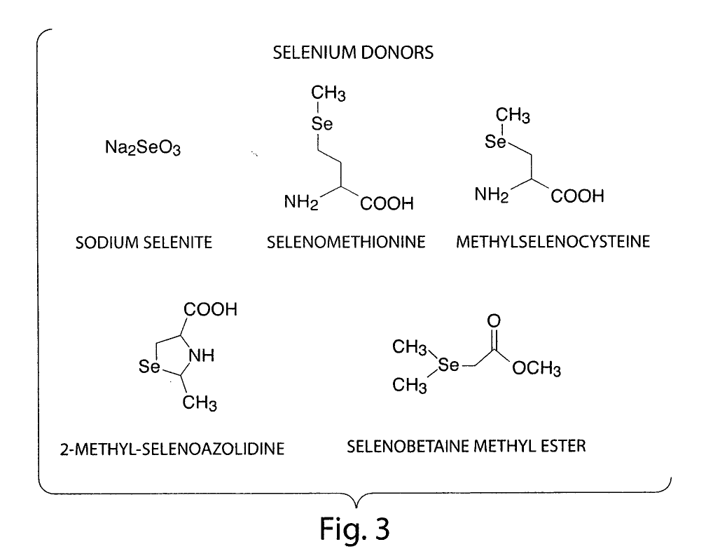

It is to be understood that the drawings are illustrative only and are not required for enablement of the invention.

### DETAILED DESCRIPTION OF THE INVENTION

Minimizing the toxicity of ROS generation requires not only the dismutation of superoxide, but also and importantly the reduction of peroxides and peroxynitrite. Peroxide elimination requires adequate peroxidase activity and adequate reducing equivalents in the form of reduced glutathione. Peroxynitrite formation should theoretically be attenuated by decreasing the steady-state concentration of superoxide. However, owing to the very rapid kinetics governing the reaction between nitric oxide and superoxide, peroxynitrite formation competes favorably with superoxide dismutation and, thus, its formation is critically dependent on nitric oxide flux. Nitric oxide production is comparatively high in the setting of inflammation-mediated inducible nitric oxide synthase expression. It is also important to note that hydroxide and hydroxyl radical are very potent ROS and likely account for much of the oxidative injury accompanying several conditions.

These biochemical arguments suggested to the applicant that the use of a SOD mimetic alone may not only fail to improve ROS-mediated injury but, rather, enhance ROS-mediated injury should peroxidase-dependent mechanisms be insufficient to meet the increased peroxide flux.

A major determinant of peroxidase activity (and peroxynitrite reductase activity, as well) is GSH peroxidase-1 (GPx-1) which is a ubiquitous selenocysteine containing enzyme. Catalase can reduce hydrogen peroxide to water and does so with a very high catalytic efficiency. However, catalase does so via a very rapid k_{cat} despite a weak K_{M}, and cannot reduce lipid peroxides. GPx-1, while having a lower catalytic efficiency, has a tighter K_{M} for substrate than does catalase and, thus, reduces lower fluxes of peroxides and does so in different compartments in the cell. In addition, GPx-1 can also eliminate lipid peroxides.

GPx-1 expression requires adequate dietary selenium. Most individuals will manifest enhanced glutathione peroxidase activity in serum or erythrocytes when supplemented with a selenium source, indicating that maximal selenium uptake is not common in most populations. This is not to say that most individuals are selenium-deficient rather, suboptimal expression of selenocysteine-containing proteins (of which there are approximately 20 in mammals) is the norm and may be inadequate for optimal response to treatment with a SOD mimetic.

Applicant believes that coupling the administration of a SOD mimetic with a selenium compound would be beneficial to minimize overall ROS generation and their adverse effects. The present invention is based, in part, on applicant's surprising and unexpected discovery that the combination of SOD mimetics and Se compounds results in a synergistic inhibition of ROS generation.

Thus, the invention involves, in some aspects, administering an effective amount of a SOD mimetic and a Se compound to a subject having an inflammatory, an autoimmune, a vascular or a cardiovascular condition to treat the subject.

The term “treatment” or “treat” is intended to include prophylaxis, amelioration, prevention or cure of a condition. Treatment after a condition has started aims to reduce, ameliorate or altogether eliminate the condition, and/or one or more of its associated symptoms, or prevent it from becoming worse. Treatment of subjects before a condition has started (i.e., prophylactic treatment) aims to reduce the risk of developing the condition and/or lessen its severity if the condition later develops. As used herein, the term “prevent” refers to the prophylactic treatment of subjects who are at risk of developing a condition which treatment results in a decrease in the probability that the subject will develop the condition, or results in an increase in the probability that the condition is less severe than it would have been absent the treatment.

A “subject” shall mean a human or animal including but not limited to a dog, cat, horse, cow, pig, sheep, goat, chicken, rodent, e.g., rats and mice, primate, e.g., monkey, and fish. Preferred subjects are human subjects. The human subject may be a pediatric, adult or a geriatric subject.

The invention involves methods for treating a subject with an inflammatory condition. Inflammation is the body's response to injury, infection, or molecules perceived by the immune system as foreign. Inflammation is characterized by pain, swelling and altered function of the affected tissue. Although the ability to mount an inflammatory response is essential for survival, the ability to control inflammation is also necessary for health. Excessive or uncontrolled inflammation results in a vast array of inflammatory conditions. Examples of inflammatory conditions include allergic rhinitis, ankylosing spondilitis, arthritis, asthma, Behcet syndrome, bursitis, chronic obstructive pulmonary disease (COPD), Churg-Strauss syndrome, dermatitis, gout, Henoch-Schonlein purpura, inflammatory bowel disease (Crohn's disease or ulcerative colitis), inflammatory neuropathy, Kawasaki disease, myositis, neuritis, pericarditis, polyarteritis nodosa, polymyalgia rheumatica, prostatitis, psoriasis, radiation injury, sarcoidosis, shock, systemic inflammatory response syndrome (SIRS), Takayasu's arteritis, temporal arteritis, thromboangiitis obliterans (Buerger's disease), vasculitis, and Wegener's granulomatosus.

The methods of the invention may also be used to treat a subject with an autoimmune condition. Autoimmune conditions are conditions caused by an immune response against the body's own tissues. The immune system protects the body from potentially harmful substances (antigens) such as microorganisms, toxins, cancer cells, and foreign blood or tissues from another person or species. Antigens are destroyed by the immune response, which includes production of antibodies and sensitized lymphocytes. Autoimmune conditions develop when the immune system destroys normal body tissues. Autoimmune conditions result in destruction of one or more types of body tissues, abnormal growth of an organ, or changes in organ function. The autoimmune condition may affect only one organ or tissue type or may affect multiple organs and tissues. Examples of organs and tissues affected by autoimmune conditions include but are not limited to blood, blood vessels, connective tissues, muscles, joints, skin, and endocrine glands (e.g., the thyroid or pancreas). Examples of autoimmune (or autoimmune-related) conditions include: Addison's disease, chronic thyroiditis, dermatomyositis, Grave's disease, Hashimoto's thyroiditis, hypersensitivity pneumonitis, insulin-dependent diabetes mellitus (type I diabetes), insulin-independent diabetes mellitus (type II diabetes), multiple sclerosis, myasthenia gravis, organ transplantation, pernicious anemia, Reiter's syndrome, rheumatoid arthritis, Sjogren's syndrome, systemic lupus erythematosis (SLE), thyroiditis, and urticaria.

The methods of the invention can also be used to treat a subject with a vascular or a cardiovascular condition. Vascular conditions are conditions that involve the blood vessels (arteries and veins). Cardiovascular conditions are conditions that involve the heart and the blood vessels associated with the heart.

Examples of vascular conditions include: ischemia-reperfusion injury, diabetic retinopathy, diabetic nephropathy, renal fibrosis, hypertension, atherosclerosis, arteriosclerosis, atherosclerotic plaque, atherosclerotic plaque rupture, cerebrovascular accident (stroke), transient ischemic attack (TIA), peripheral artery disease, arterial occlusive disease, vascular aneurysm, ischemia, ischemic ulcer, heart valve stenosis, heart valve regurgitation and intermittent claudication.

Ischemia-reperfusion injury refers to damage to a tissue caused when blood supply returns to the tissue after a period of ischemia. It is believed that the absence of oxygen and nutrients from blood creates a condition in which the restoration of circulation results in inflammation and oxidative damage from the oxygen rather than restoration of normal function.

Examples of cardiovascular conditions include coronary artery disease, ischemic cardiomyopathy, myocardial ischemia, and ischemic or post-myocardial ischemia revascularization.

The invention also involves treating a subject at risk for developing a cardiovascular condition. The degree of risk of a cardiovascular condition depends on the multitude and the severity or the magnitude of the risk factors that the subject has. Risk charts and prediction algorithms are available for assessing the risk of cardiovascular conditions in a human subject based on the presence and severity of risk factors. One commonly used algorithm for assessing the risk of a cardiovascular condition in a human subject based on the presence and severity of risk factors is the Framingham Heart Study risk prediction score. A human subject is at an elevated risk of having a cardiovascular condition if the subject's 10-year calculated Framingham Heart Study risk score is greater than 10%.

Another method for assessing the risk of a cardiovascular event in a human subject is a global risk score that incorporates a measurement of a level of a marker of systemic inflammation, such as CRP, into the Framingham Heart Study risk prediction score. Other methods of assessing the risk of a cardiovascular event in a human subject include coronary calcium scanning, cardiac magnetic resonance imaging, and/or magnetic resonance angiography.

The invention involves methods of treatment comprising administering an effective amount of a superoxide dismutase (SOD) mimetic and a Se compound. As used herein the term “SOD mimetic” encompasses compounds that convert or catalyze the conversion of superoxide anions into hydrogen peroxide and molecular oxygen. SOD mimetics include but are not limited to manganese-tetrakis (4-benzoic acid) porphyrin (MnTBAP) M40403 (Samlowski WE et al., Nature Medicine 2003;9:750-755), metalloporphyrins, manganese salen complexes, nitroxides, manganese (II) (pentaazamacrocyclic ligand)-based complexes and organic ligands having a pentaazacyclopentadecane portion and a chelated transition metal ion, preferably manganese or iron. SOD mimetics include catalysts containing short-chain polypeptides (e.g., under 15 amino acids), and macrocyclic structures derived from amino acids, as the organic ligand. In some embodiments, SOD mimetics include pentaaza-macrocyclic ligand compounds, such as copper, manganese(II), manganese (III), iron(II) and iron(III) chelates of pentaazacyclopentadecane compounds.

The term “Se compound” encompasses the element Selenium (Se) and sources of the element (e.g., dietary sources such as cereals, grains and vegetables) as well as compounds that contain Se. Se compounds are molecules that are able to catalyze the oxidation of thiols and to produce superoxide simultaneously. Examples of Se compounds include but are not limited to sodium selenite, selenomethionine, methylselenocysteine, 2-methyl-selenoazolidine, selenobetaine methyl ester, selenocysteine, selenobetaine, and selenoazolidine.

The SOD mimetic(s) and the Se compound(s) are administered in an amount effective to treat the condition in the subject. An effective amount is a dosage of the therapeutic agent(s) sufficient to provide a medically desirable response. For example the desirable response may be inhibiting the progression of the condition. This may involve only slowing the progression of the condition temporarily, although more preferably, it involves halting the progression of the condition permanently. This can be monitored by routine diagnostic methods known to those of ordinary skill in the art.

It should be understood that the therapeutic agents of the invention are used to treat or prevent the condition, that is, they may be used prophylactically in subjects at risk of developing the condition. Thus, an effective amount is that amount which can lower the risk of, lessen the severity of, or perhaps prevent altogether the development of the condition.

It will be recognized when the therapeutic agents are used in acute circumstances, they are used to prevent one or more medically undesirable results that typically flow from such adverse events

The factors involved in determining an effective amount are well known to those of ordinary skill in the art and can be addressed with no more than routine experimentation. It is generally preferred that a maximum dose of the therapeutic agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The SOD mimetic(s) and the Se compound(s) may be administered alone, in a pharmaceutical composition or combined with other therapeutic agents or regimens. Optionally other therapeutic agent(s) may be administered simultaneously or sequentially. When the other therapeutic agent(s) are administered simultaneously they can be administered in the same or separate formulations, but are administered at the same time. The other therapeutic agent(s) may be administered sequentially with one another and with the SOD mimetic(s) and the Se compound(s) when the administration of the other therapeutic agent(s) and the SOD mimetic(s) and the Se compound(s) are temporally separated. The separation in time between the administration of these compounds may be a matter of minutes or it may be longer.

In some embodiments the SOD mimetic(s) and the Se compound(s) may be administered with an anti-inflammatory agent that is not a SOD mimetic or a Se compound. Examples of anti-inflammatory agents that are not SOD mimetics or Se compounds include but are not limited to Alclofenac, Alclometasone Dipropionate, Algestone Acetonide, Alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Cyclooxygenase-2 (COX-2) inhibitor, Deflazacort, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lornoxicam, Loteprednol Etabonate, Meclofenamate Sodium, Meclofenamic Acid, Meclorisone Dibutyrate, Mefenamic Acid, Mesalamine, Meseclazone, Methylprednisolone Suleptanate, Momiflumate, Nabumetone, Naproxen, Naproxen Sodium, Naproxol, Nimazone, Olsalazine Sodium, Orgotein, Orpanoxin, Oxaprozin, Oxyphenbutazone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Sudoxicam, Sulindac, Suprofen, Talmetacin, Talniflumate, Talosalate, Tebufelone, Tenidap, Tenidap Sodium, Tenoxicam, Tesicam, Tesimide, Tetrydamine, Tiopinac, Tixocortol Pivalate, Tolmetin, Tolmetin Sodium, Triclonide, Triflumidate, Zidometacin, and Zomepirac Sodium. In some preferred embodiments the anti-inflammatory agent that is not a SOD mimetic or a Se compound is Naproxen or Ibuprofen.

In some embodiments the SOD mimetic(s) and the Se compound(s) may be administered with an autoimmune condition agent that is not a SOD mimetic or a Se compound. The autoimmune condition agent may be an immunosuppressive agent. Examples of immunosuppressive agents include but are not limited to steroids, a calcineurine inhibitors, monoclonal antibodies and polyclonal antibodies. The steroid may be a corticosteroid. Examples of corticosteroids include prednisone, methylprednisone, and methylprednisolone. Examples of calcineurine inhibitors include cyclosporine and tacrolimus. Examples of monoclonal antibodies include anti-CD3 antibody (Muromonab-CD3; Orthoclone OKT3®) and anti-IL-2R alpha chain (p55). Examples of polyclonal antibodies include anti-thymocyte globulin-equine (Atgam®) and anti-thymocyte globulin-rabbit (RATG thymoglobulin®). The immunosuppressive agent also may be, for example, sulfasalazine, FK-506, methoxsalen, thalidomide, mycophenolate mofetil (MMF), azathioprine or sirolimus. In some preferred embodiments, the immunosuppressive agent is methylprednisolone or cyclosprorine.

In some embodiments the SOD mimetic(s) and the Se compound(s) may be administered with a vascular agent or a cardiovascular agent that is not a SOD mimetic or a Se compound. Examples of vascular agents or cardiovascular agents that are not SOD mimetics or Se compounds include but are not limited to anti-lipemic agents, anti-thrombotic agents, fibrinolytic agents, anti-platelet agents, direct thrombin inhibitors, glycoprotein IIb/IIIa receptor inhibitors, agents that bind to cellular adhesion molecules and inhibit the ability of white blood cells to attach to such molecules (e.g., anti-cellular adhesion molecule antibodies), alpha-adrenergic blockers, beta-adrenergic blockers, angiotensin system inhibitor, anti-arrhythmics, calcium channel blockers, diuretics, inotropic agents, vasodilators, vasopressors, and/or any combinations thereof.

Anti-lipemic agents are agents that reduce total cholesterol, reduce LDLC, reduce triglycerides, and/or increase HDLC. Anti-lipemic agents include statins and non-statin anti-lipemic agents, and/or combinations thereof. Statins are a class of medications that have been shown to be effective in lowering human total cholesterol, LDLC and triglyceride levels and are widely used to reduce atherosclerotic morbidity and mortality.

Examples of statins include, but are not limited to, simvastatin (Zocor), lovastatin (Mevacor), pravastatin (Pravachol), fluvastatin (Lescol), atorvastatin (Lipitor), cerivastatin (Baycol), rosuvastatin (Crestor), pitivastatin and numerous others described in U.S. Pat. No. 4,444,784, U.S. Pat. No. 4,231,938, U.S. Pat. No. 4,346,227, U.S. Pat. No. 4,739,073, U.S. Pat. No. 5,273,995, U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402.

Examples of statins already approved for use in humans include atorvastatin, cerivastatin, fluvastatin, pravastatin, simvastatin and rosuvastatin. The reader is referred to the following references for further information on HMG-CoA reductase inhibitors: Drugs and Therapy Perspectives (May 12, 1997), 9:1-6; Chong (1997) Pharmacotherapy 17:1157-1177; Kellick (1997) Formulary 32: 352; Kathawala (1991) Medicinal Research Reviews, 11:121-146; Jahng (1995) Drugs of the Future 20:387-404, and Current Opinion in Lipidology, (1997), 8, 362-368. Another statin drug of note is compound 3a (S-4522) in Watanabe (1997) Bioorganic and Medicinal Chemistry 5:437-444.

Non-statin anti-lipemic agents include but are not limited to fibric acid derivatives (fibrates), bile acid sequestrants or resins, nicotinic acid agents, cholesterol absorption inhibitors, acyl-coenzyme A: cholesterol acyl transferase (ACAT) inhibitors, cholesteryl ester transfer protein (CETP) inhibitors, LDL receptor antagonists, famesoid X receptor (FXR) antagonists, sterol regulatory binding protein cleavage activating protein (SCAP) activators, microsomal triglyceride transfer protein (MTP) inhibitors, squalene synthase inhibitors, and peroxisome proliferation activated receptor (PPAR) agonists.

Examples of fibric acid derivatives include but are not limited to gemfibrozil (Lopid), fenofibrate (Tricor), clofibrate (Atromid) and bezafibrate.

Examples of bile acid sequestrants or resins include but are not limited to colesevelam (WelChol), cholestyramine (Questran or Prevalite) and colestipol (Colestid), DMD-504, GT-102279, HBS-107 and S-8921.

Examples of nicotinic acid agents include but are not limited to niacin and probucol.

Examples of cholesterol absorption inhibitors include but are not limited to ezetimibe (Zetia).

Examples of ACAT inhibitors include but are not limited to Avasimibe, CI-976 (Parke Davis), CP-113818 (Pfizer), PD-138142-15 (Parke Davis), F1394, and numerous others described in U.S. Pat. Nos. 6,204,278, 6,165,984, 6,127,403, 6,063,806, 6,040,339, 5,880,147, 5,621,010, 5,597,835, 5,576,335, 5,321,031, 5,238,935, 5,180,717, 5,149,709, and 5,124,337.

Examples of CETP inhibitors include but are not limited to Torcetrapib, CP-529414, CETi-1, JTT-705, and numerous others described in U.S. Pat. Nos. 6,727,277, 6,723,753, 6,723,752, 6,710,089, 6,699,898, 6,696,472, 6,696,435, 6,683,099, 6,677,382, 6,677,380, 6,677,379, 6,677,375, 6,677,353, 6,677,341, 6,605,624, 6,586,448, 6,521,607, 6,482,862, 6,479,552, 6,476,075, 6,476,057, 6,462,092, 6,458,852, 6,458,851, 6,458,850, 6,458,849, 6,458,803, 6,455,519, 6,451,830, 6,451,823, 6,448,295, 5,512,548.

One example of an FXR antagonist is Guggulsterone. One example of a SCAP activator is GW532 (GlaxoSmithKline).

Examples of MTP inhibitors include but are not limited to Implitapide and R-103757.

Examples of squalene synthase inhibitors include but are not limited to zaragozic acids.

Examples of PPAR agonists include but are not limited to GW-409544, GW-501516, and LY-510929.

Anti-thrombotic agents and/or fibrinolytic agents include tissue plasminogen activator (e.g., Activase, Alteplase) (catalyzes the conversion of inactive plasminogen to plasmin. This may occur via interactions of prekallikrein, kininogens, Factors XII, XIIIa, plasminogen proactivator, and tissue plasminogen activator TPA) Streptokinase, Urokinase, Anisoylated Plasminogen-Streptokinase Activator Complex, Pro-Urokinase, (Pro-UK), rTPA (alteplase or activase; r denotes recombinant), rPro-UK, Abbokinase, Eminase, Sreptase Anagrelide Hydrochloride, Bivalirudin, Dalteparin Sodium, Danaparoid Sodium, Dazoxiben Hydrochloride, Efegatran Sulfate, Enoxaparin Sodium, Ifetroban, Ifetroban Sodium, Tinzaparin Sodium, retaplase, Trifenagrel, Warfarin, Dextrans, aminocaproic acid (Amicar), and tranexamic acid (Amstat).

Anti-platelet agents include Clopridogrel, Sulfinpyrazone, Aspirin, Dipyridamole, Clofibrate, Pyridinol Carbamate, PGE, Glucagon, Antiserotonin drugs, Caffeine, Theophyllin Pentoxifyllin, Ticlopidine, and Anagrelide.

Direct thrombin inhibitors include hirudin, hirugen, hirulog, agatroban, PPACK, and thrombin aptamers.

Glycoprotein IIb/IIIa receptor Inhibitors are both antibodies and non-antibodies, and include but are not limited to ReoPro (abcixamab), lamifiban, and tirofiban.

Agents that bind to cellular adhesion molecules and inhibit the ability of white blood cells to attach to such molecules include polypeptide agents. Such polypeptides include polyclonal and monoclonal antibodies, prepared according to conventional methodology. Such antibodies already are known in the art and include anti-ICAM 1 antibodies as well as other such antibodies.

Examples of alpha-adrenergic blockers include: doxazocin, prazocin, tamsulosin, and tarazosin.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol,7-(2-hydroxy-3-t-butylaminpropoxy)phthalide. The above-identified compounds can be used as isomeric mixtures, or in their respective levorotating or dextrorotating form.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin I and angiotensin II are synthesized by the enzymatic renin-angiotensin pathway. The synthetic process is initiated when the enzyme renin acts on angiotensinogen, a pseudoglobulin in blood plasma, to produce the decapeptide angiotensin I. Angiotensin I is converted by angiotensin converting enzyme (ACE) to angiotensin II (angiotensin-[1-8] octapeptide). The latter is an active pressor substance which has been implicated as a causative agent in several forms of hypertension in various mammalian species, e.g., humans.

Angiotensin (renin-angiotensin) system inhibitors are compounds that act to interfere with the production of angiotensin II from angiotensinogen or angiotensin I or interfere with the activity of angiotensin II. Such inhibitors are well known to those of ordinary skill in the art and include compounds that act to inhibit the enzymes involved in the ultimate production of angiotensin II, including renin and ACE. They also include compounds that interfere with the activity of angiotensin II, once produced. Examples of classes of such compounds include antibodies (e.g., to renin), amino acids and analogs thereof (including those conjugated to larger molecules), peptides (including peptide analogs of angiotensin and angiotensin I), pro-renin related analogs, etc. Among the most potent and useful renin-angiotensin system inhibitors are renin inhibitors, ACE inhibitors, and angiotensin II antagonists. Preferred renin-angiotensin system inhibitors are renin inhibitors, ACE inhibitors, and angiotensin II antagonists.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid. Stability can be enhanced by other replacements that slow degeneration in vivo. Examples of angiotensin II receptor antagonists include but are not limited to: Candesartan (Alacand), Irbesartan (Avapro), Losartan (Cozaar), Telmisartan (Micardis), and Valsartan (Diovan). Other examples of angiotensin II antagonists include: peptidic compounds (e.g., saralasin, [(Sar¹)(Val⁵)(Ala⁸)] angiotensin -(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo [4,5-c] pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1, 3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl] 1H-imidazole-5-yl[methylane]-2-thiophenepropanoic acid, Smith Kline Beecham Pharmaceuticals, Pa.); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G. D. Searle and Company).

Angiotensin converting enzyme (ACE), is an enzyme which catalyzes the conversion of angiotensin I to angiotensin II. ACE inhibitors include amino acids and derivatives thereof, peptides, including di and tri peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di and tri peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

Calcium channel blockers are a chemically diverse class of compounds having important therapeutic value in the control of a variety of diseases including several vascular and cardiovascular conditions, such as hypertension, angina, and cardiac arrhythmias (Fleckenstein, Cir. Res. v. 52, (suppl. 1), p.13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Eaton, Pa., p.963 (1995)). Most of the currently available calcium channel blockers belong to one of three major chemical groups of drugs, the dihydropyridines, such as nifedipine, the phenyl alkyl amines, such as verapamil, and the benzothiazepines, such as diltiazem. Other calcium channel blockers include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Diuretics include but are not limited to: carbonic anhydrase inhibitors, loop diuretics, potassium-sparing diuretics, thiazides and related diuretics.

Vasodilators include but are not limited to coronary vasodilators and peripheral vasodilators.

Vasopressors are agents that produce vasoconstriction and/or a rise in blood pressure. Vasopressors include but are not limited to: dopamine, ephedrine, epinephrine, Methoxamine HCl (Vasoxyl), norepinephrine, phenylephrine, phenylephrine HCl (Neo-Synephrine), and Metaraminol. In some preferred embodiments, the vasopressor is dopamine or norepinehrine.

The pharmaceutical compositions used in the methods of the invention are preferably sterile and contain effective amounts of the SOD mimetic(s) and the Se compound(s) for producing the desired response in a unit of weight or volume suitable for administration to a subject. The doses of pharmacological agent(s) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

In some embodiments, the preferred SOD mimetic is manganese-tetrakis (4-benzoic acid) porphyrin (MnTBAP) administered at 5-10 mg/kg/day or the related compound M40403 administered at 1-5 mg/kg/day. In some embodiments, the preferred Se compound is Se-methylselenocysteine administered at 0.2-6.0 mg/kg/day (˜0.06-2 mg selenium/kg/day).

Various modes of administration are known to those of ordinary skill in the art which effectively deliver the pharmacological agents of the invention to a desired tissue, cell, or bodily fluid. The administration methods are discussed elsewhere in the application. The invention is not limited by the particular modes of administration disclosed herein. Standard references in the art (e.g., Remington's Pharmaceutical Sciences, 20th Edition, Lippincott, Williams and Wilkins, Baltimore Md., 2001) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration and the like vary from those presented herein. Administration of pharmacological agents of the invention to mammals other than humans, e.g., for testing purposes or veterinary therapeutic purposes, is carried out under substantially the same conditions as described above. It will be understood by one of ordinary skill in the art that this invention is applicable to both human and animal diseases. Thus, this invention is intended to be used in husbandry and veterinary medicine as well as in human therapeutics.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the compounds may be in powder form for constitution with a suitable vehicle (e.g., saline, buffer, or sterile pyrogen-free water) before use.

Compositions suitable for oral administration may be presented as discrete units, such as capsules, tablets, pills, lozenges, each containing a predetermined amount of the active compound(s) (e.g., SOD mimetic and/or Se compound). Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the SOD mimetic(s) and/or the Se compound(s) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the SOD mimetic(s) and the Se compound(s) may be formulated (such as by liposome or microsphere encapsulation) and then further contained within an edible product, such as a refrigerated beverage containing colorants and flavoring agents.

One may dilute or increase the volume of the therapeutic agent with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the SOD mimetic(s) and/or the Se compound(s) either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of SOD mimetic(s) and/or the Se compound(s). The SOD mimetic(s) and the Se compound(s) are delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins,” Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the SOD mimetic(s) and the Se compound(s). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified SOD mimetic(s) and/or the Se compound(s) may also be prepared in different formulations depending on the type of chemical modification or the type of device employed.

Formulations suitable for use with a nebulizer, either jet or ultrasonic, will typically comprise the SOD mimetic(s) and/or the Se compound(s) dissolved in water at a concentration of about 0.1 to 25 mg of SOD mimetic(s) and the Se compound(s) per mL of solution. The formulation may also include a buffer and a simple sugar (e.g., for stabilization and regulation of osmotic pressure). The nebulizer formulation may also contain a surfactant, to reduce or prevent surface induced aggregation of the SOD mimetic(s) and the Se compound(s) caused by atomization of the solution in forming the aerosol.

Formulations for use with a metered-dose inhaler device will generally comprise a finely divided powder containing the SOD mimetic(s) and/or the Se compound(s) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the SOD mimetic(s) and/or the Se compound(s) and may also include a bulking agent, such as lactose, sorbitol, sucrose, or mannitol in amounts which facilitate dispersal of the powder from the device, e.g., 50 to 90% by weight of the formulation. The SOD mimetic(s) and/or the Se compound(s) should most advantageously be prepared in particulate form with an average particle size of less than 10 μm (or microns), most preferably 0.5 to 5 μm, for most effective delivery to the distal lung.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The SOD mimetic(s) and/or the Se compound(s) and optionally other therapeutics may be administered per se or in the form of a pharmaceutically acceptable salt.

The therapeutic agent(s), including SOD mimetic(s) and/or the Se compound(s) may be provided in particles. Particles as used herein means nano or microparticles (or in some instances larger) which can consist in whole or in part of the SOD mimetic(s) and/or the Se compound(s) or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the SOD mimetic(s) and/or the Se compound(s) in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

For topical administration to the eye, nasal membranes, mucous membranes or to the skin, the SOD mimetic(s) and/or the Se compound(s) may be formulated as ointments, creams or lotions, or as a transdermal patch or intraocular insert or iontophoresis. For example, ointments and creams can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel,” issued to Mueller, D., et al., for a description of a pharmaceutically acceptable gel-based topical carrier.)

In general, the SOD mimetic(s) and/or the Se compound(s) are present in a topical formulation in an amount ranging from about 0.01% to about 30.0% by weight, based upon the total weight of the composition. Preferably, the SOD mimetic(s) and/or the Se compound(s) are present in an amount ranging from about 0.5 to about 30% by weight and, most preferably, the SOD mimetic(s) and/or the Se compound(s) are present in an amount ranging from about 0.5 to about 10% by weight. In one embodiment, the compositions of the invention comprise a gel mixture to maximize contact with the surface of the localized pain and minimize the volume and dosage necessary to alleviate the localized pain. GELFOAM® (a methylcellulose-based gel manufactured by Upjohn Corporation) is a preferred pharmaceutically acceptable topical carrier. Other pharmaceutically acceptable carriers include iontophoresis for transdermal drug delivery.

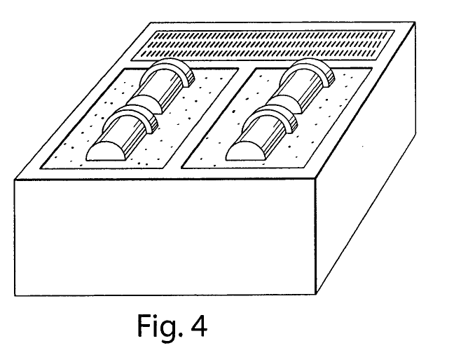

The invention also contemplates the use of kits. In some aspects of the invention, the kit can include a pharmaceutical preparation vial, a pharmaceutical preparation diluent vial, and the SOD mimetic(s) and the Se compound(s). The vial containing the diluent for the pharmaceutical preparation is optional. The diluent vial contains a diluent such as physiological saline for diluting what could be a concentrated solution or lyophilized powder of the SOD mimetic(s) and the Se compound(s). The instructions can include instructions for mixing a particular amount of the diluent with a particular amount of the concentrated pharmaceutical preparation, whereby a final formulation for injection or infusion is prepared. The instructions may include instructions for treating a subject with an effective amount of the SOD mimetic(s) and the Se compound(s). It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized. One example of a kit is illustrated in FIG. 4.

The present invention is further illustrated by the following Example, which in no way should be construed as further limiting. The entire contents of all of the references (including literature references, issued patents, published patent applications) cited throughout this application are hereby expressly incorporated by reference.

### EXAMPLES

### Example 1

Hearts were isolated from rats and perfused in the Langendorff mode as previously described (Jain et al., Am J Physiol. 2001; 280:H569-H575; Lim et al., Am J Physiol. 1999; 277:H2083-H2090). Briefly, animals were injected intraperitoneally with heparin (10,000 U/kg) and anesthetized with a mixture of ketamine (150 mg/kg) and xylazine (15 mg/kg). The thorax was rapidly opened and the heart excised and arrested in ice-cold saline. A short perfusion cannula was inserted into the aortic root to initiate retrograde perfusion. The hearts were perfused with Krebs-Henseleit buffer (in mmol/L: NaCl 118, KCl 4.7, CaCl₂ 1.8, KH₂PO₄ 1.2, MgSO₄ 1.2, NaHCO₃ 24.0, and glucose 10.0) equilibrated with 95% O₂/5% CO₂ to yield a pH of 7.4. A thin cannula was pierced through the apex of the left ventricle (LV) to vent thebesian drainage. A ventricular balloon, composed of polyvinyl chloride film and connected to a polyethylene tube, was inserted into the LV through the mitral valve via an incision in the left atrium. The balloon was connected to a pressure transducer (Statham P23Db, Gould) for recording of LV pressures. The balloon was inflated with saline to adjust the end-diastolic pressure (EDP) to 10 mm Hg, and the balloon volume was held constant for the duration of the experiment. Hearts were paced (Grass Instruments) with platinum wires placed on the epicardial surface of the right ventricle. Coronary perfusion pressure was held constant during the duration of the experiment at 80 mm Hg. An inline ultrasonic flow probe (Transonics Systems Inc) was positioned immediately above the aortic cannula to measure coronary flow. Systolic pressure, EDP, and coronary flow were collected online by use of a commercially available data acquisition system (PowerLab ADInstruments). Developed pressure (DevP) (the difference between systolic and diastolic pressures) and EDP were used as indices of contractile and diastolic function, respectively.

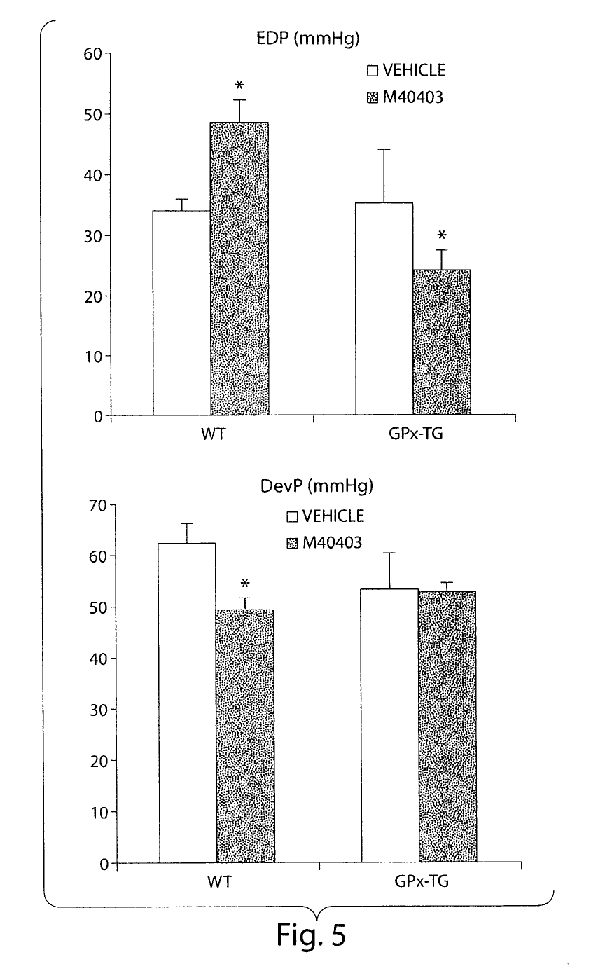

During 20 minutes of baseline stabilization, all hearts were maintained at 37° C. and paced at 7 Hz. Hearts were subsequently subjected to 15 minutes of zero-flow ischemia followed by 30 minutes of reperfusion with or without a reperfusion solution containing 23 μM of the SOD mimetic M40403. The EDP was higher and DevP was lower in the wild-type (WT) rats with this treatment compared with vehicle control. However, when glutathione peroxidase-1, the selenocysteine-containing antioxidant protein, was overexpressed as a transgene (GPx-TG) with added dietary selenium, M40403 administration was associated with improved EDP (i.e., lower EDP) and no change DevP compared with vehicle control (FIG. 5). These data support our hypothesis that administration of SOD mimetics alone will not improve ventricular performance (i.e., lower EDP and increase DevP) following ischemia-reperfusion, while the combination of the SOD mimetic and a Se compound significantly improves performance.

### Example 2

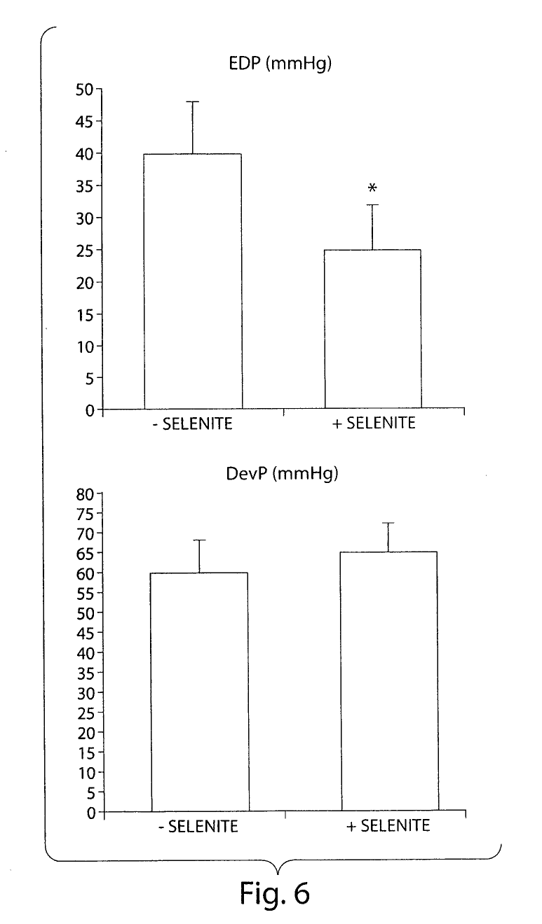

Hearts were treated first for 2 weeks with or without Se supplementation (as sodium selenite, 50 μg/kg/day), after which they were subjected to no-flow ischemia-reperfusion with 23 μM of the SOD mimetic M40403 added to the perfusate. EDP and DevP (as measures of ventricular performance) were monitored over time following restoration of perfusion. The effect of M40403 alone was compared to the effect of the combination of the M40403 and the sodium selenite on ventricular performance. M40403 administration with sodium selenite was associated with improved EDP (i.e., lower EDP) compared to M40403 administration without sodium selenite (FIG. 6).

### Example 3

Hearts are treated first for 2 days with or without Se-methylselenocysteine (1 mg/kg/day), after which they are subjected to no-flow ischemia-reperfusion with 23 μM of the SOD mimetic M40403 added to the perfusate (or with another SOD mimetic at a final perfusate concentration of 5-25 μmol/L). EDP and DevP (as measures of ventricular performance) are monitored over time following restoration of perfusion. The effect of SOD-mimetic alone is compared to the effect of the combination of the SOD-mimetic and the Se-methylselenocysteine on ventricular performance.

We claim:

1. A method for treating a subject having an inflammatory condition comprising:
administering to the subject an effective amount of a SOD mimetic and a Se compound to treat the subject.

2. The method of claim 1, wherein the SOD mimetic is, manganese-tetrakis (4-benzoic acid) porphyrin (MnTBAP), M40403, a manganese metalloporphyrin, a manganese salen complex, a nitroxide, or a manganese (II) (pentaazamacrocyclic ligand)-based complex.

3. The method of claim 1, wherein the Se compound is sodium selenite, selenomethionine, methylselenocysteine, 2-methyl-selenoazolidine, selenobetaine methyl ester, selenocysteine, selenobetaine, or selenoazolidine.

4. The method of claim 1, wherein the inflammatory condition is allergic rhinitis, ankylosing spondilitis, arthritis, asthma, Behcet syndrome, bursitis, chronic obstructive pulmonary disease (COPD), Churg-Strauss syndrome, dermatitis, gout, Henoch-Schonlein purpura, inflammatory bowel disease (Crohn's disease or ulcerative colitis), inflammatory neuropathy, Kawasaki disease, myositis, neuritis, pericarditis, polyarteritis nodosa, polymyalgia rheumatica, prostatitis, psoriasis, radiation injury, sarcoidosis, shock, systemic inflammatory response syndrome (SIRS), Takayasu's arteritis, temporal arteritis, thromboangiitis obliterans (Buerger's disease), vasculitis, or Wegener's granulomatosus.

5-7. (canceled)

8. A method for treating a subject having an autoimmune condition comprising:
administering to the subject an effective amount of a SOD mimetic and a Se compound to treat the subject.

9. The method of claim 8, wherein the SOD mimetic is manganese-tetrakis (4-benzoic acid) porphyrin (MnTBAP), M40403, a manganese metalloporphyrin, a manganese salen complex, a nitroxide, or a manganese (II) (pentaazamacrocyclic ligand)-based complex.

10. The method of claim 8, wherein the Se compound is sodium selenite, selenomethionine, methylselenocysteine, 2-methyl-selenoazolidine, selenobetaine methyl ester, selenocysteine, selenobetaine, or selenoazolidine.

11. The method of claim 8, wherein the autoimmune condition is Addison's disease, chronic thyroiditis, dermatomyositis, Grave's disease, Hashimoto's thyroiditis, hypersensitivity pneumonitis, insulin-dependent diabetes mellitus (type I diabetes), insulin-independent diabetes mellitus (type II diabetes), multiple sclerosis, myasthenia gravis, organ transplantation, pernicious anemia, Reiter's syndrome, rheumatoid arthritis, Sjogren's syndrome, systemic lupus erythematosis (SLE), thyroiditis, or urticaria.

12-15. (canceled)

16. A method for treating a subject having a vascular or a cardiovascular condition or is at risk of developing a cardiovascular condition comprising:
administering to the subject an effective amount of a SOD mimetic and a Se compound to treat the subject.

17. (canceled)

18. The method of claim 16, wherein the SOD mimetic is manganese-tetrakis (4-benzoic acid) porphyrin (MnTBAP), M40403, a manganese metalloporphyrin, a manganese salen complex, a nitroxide, or a manganese (II) (pentaazarnacrocyclic ligand)-based complex.

19. The method of claim 16, wherein the Se compound is sodium selenite, selenomethionine, methylselenocysteine, 2-methyl-selenoazolidine, selenobetaine methyl ester, selenocysteine, selenobetaine, or selenoazolidine.

20. The method of claim 16, wherein the vascular condition is ischemia-reperfusion injury.

21. (canceled)

22. The method of claim 16, wherein the vascular condition is diabetic retinopathy, diabetic nephropathy, renal fibrosis, hypertension, atherosclerosis, arteriosclerosis, atherosclerotic plaque, atherosclerotic plaque rupture, cerebrovascular accident (stroke), transient ischemic attack (TIA), peripheral artery disease, arterial occlusive disease, vascular aneurysm, ischemia, ischemic ulcer, heart valve stenosis, heart valve regurgitation and intermittent claudication.

23-26. (canceled)

27. A pharmaceutical composition comprising a SOD mimetic, a Se compound, and a pharmaceutically acceptable carrier.

28. The pharmaceutical composition of claim 27, wherein the SOD mimetic and the Se-containing compound are present in an effective amount.

29. The pharmaceutical composition of claim 27, further comprising an agent that is not a SOD mimetic or a Se compound.

30. (canceled)

31. The pharmaceutical composition of claim 30, wherein the antioxidant agent is: Vitamin A, Vitamin C, Vitamin E, ubiquinone (Coenzyme Q-10), allopurinol, alpha lipoic acid (ALA), oxothiazolidine-4-carboxylate or glutathione monoethylester or a carotenoid.

32. The pharmaceutical composition of claim 31, wherein the carotenoid is alphacarotene, betacarotene, lycopene, lutein, cryptoxanthin, zeaxanthin, oxothiazolidine-4-carboxylate or glutathione monoethylester.

33-35. (canceled)

36. A composition comprising manganese-tetrakis (4-benzoic acid) porphyrin (MnTBAP) in a unit dosage of about 350 mg and a Se-methylselenocysteine in a unit dosage of about 150 mg.

37. (canceled)

38. A kit comprising a SOD mimetic and a Se compound and instructions of use.

39. (canceled)
